(19) United States
(12) Patent Application Publication
(10) Pub. No.: US 2006/0264408 A1
(43) Pub. Date: Nov. 23, 2006

(54) Sod minic multifunctional compounds for treating inflammatory bowel disease

(76) Inventor: Abdullah Ibrahim Haj-Yehia, Neve Shalom (IL)

Correspondence Address: Kevin D McCarthy;Roach Brown McCarthy & Gruber, 420 Main Street, 1620 Liberty Building, Buffalo, NY, 14202, US

(21) Appl. No.: 10/547,582

(22) PCT Filed: Mar. 4, 2004
(86) PCT No.: PCT/IL04/00222

§ 371 Date: Jul. 3, 2006

Related U.S. Application Data
(60) Provisional application No. 60/451,957, filed on Mar. 6, 2003.

(51) Int. Cl. A61K 31/655; A61K 31/60; A61K 31/58; A61K 31/573; A61K 31/4184; A61K 31/385

(52) U.S. Cl. 514/150; 514/171; 514/440; 514/568; 514/159; 514/414; 514/394

(57) ABSTRACT

The present invention relates to multifunctional compounds comprising an anti-inflammatory or immunomodulatory component, and at least one SOD mimic component capable of reacting with reactive oxygen species, and to their use in treating inflammatory bowel disease comprising ulcerative colitis and Crohn's disease. Preferred anti-inflammatory or immunomodulatory components comprise derivatives of salicylic acid and steroids.

### FIELD OF THE INVENTION

The present invention relates to a novel medication for treating inflammatory bowel disease, comprising simultaneous SOD-mimetic activity and anti-inflammatory or immunomodulatory activity.

### BACKGROUND OF THE INVENTION

Ulcerative colitis (UC) and Crohn's disease (CD) are inflammatory bowel diseases (IBD) of unknown etiology, clinically characterized by pain, diarrhea, bleeding, and fatigue. Periods of exacerbations and remissions alternate, and as years pass, the chances of developing carcinoma of the colon increase. Bleeding from the ulcerated surface of the colon is chronic and at times severe, as blood vessels are eroded by the ulcerations. Continual blood loss leads to iron deficiency anemia. Nearly one million people in US alone may be afflicted, sooner or later, by IBD.

To date, only limited pharmacotherapeutic options are available for the treatment of IBD, including anti-inflammatory and immunosuppressive drugs. For routine maintenance of remission, sulfasalazine and aminosalicylates are used [Cohen R. D. et al.: Am J Gastroenterol. 95 (2000) 1263-76; Lochs H. et al.: Gastroenterology 118 (2000) 264-73]. During flare-ups, occurring despite the maintenance therapy, potent anti-inflammatory agents are required for inducing the remission, including steroids such as prednisolone, prednisone, or budesonide. Other drugs have also been tried, including metronidazole and ciprofloxacin, azathioprine and its active metabolite 6-mercaptopurine, methotrexate, infliximab, cyclosporine, mycophenolate mofetil, and agents with IL-10 and IL-11-like activity; however, none of these agents was proven to be more effective than conventional aminosalicylates and steroids.

The area of IBD is an example of unmet need at the pharmacotherapeutic level. Even if the confounding factors like misdiagnosis, occult infection, inadequate dosing or therapy duration, and poor compliance are discounted, about 30-50% of patients remain who do not respond to any therapy. It is therefore an object of this invention to provide a new medication for alleviating and treating the devastating conditions associated with IBD.

Although the initial stimulus provoking the inflammation of colon mucosa is not clear, certain chemical mechanisms are suspected to drive relapses of the disease, including superoxide-driven Fenton reaction, lipid peroxidation by low-molecular-weight ferrous/ferric complexes, chloramine generation, and free-radical-mediated carcinogen activation.

It seems that the interaction of leukocyte-generated superoxide with the relatively high concentration of low-molecular-weight chelate iron in close proximity to the sites of mucosal inflammation plays an important role in amplifying the inflammatory response and in accelerating the mucosal ulceration during flare-ups of IBD. The Fenton reaction provides the highly reactive hydroxyl radicals at physiologic pH and temperature [Smith J. B. et al.: Free Rad. Res. Commun. 8 (1990) 101-6]. Hydroxyl radicals are extremely potent oxidants capable of initiating many deleterious reactions, including lipid peroxidation and carcinogen activation [e.g., Babbs C. F.: Free Rad. Biol. Med. 8 (1990) 191-200]. It has been shown that bile pigments, abundantly present in colon contents, can combine with iron to form chelates that are highly effective in the catalysis of hydroxyl radical formation by the superoxide-driven Fenton reaction. Accordingly, it is chemically plausible that the products of free radical reactions initiated by hydroxyl radicals might participate in the pathogenesis of the disease. In the particular setting of ulcerative colitis, the hydroxyl radicals may be generated by the reaction of leukocyte-related hydrogen peroxide with ferrous heme iron derived from the red blood cells extravasated during mucosal hemorrhage [Caughey W. S. et al. in: Greenwald R. A. ed. Handbook of Methods for Oxygen Radicals Research, Boca Raton, Fla., CRC Press, pp 95-104, 1985]. Human neutrophils, stimulated to produce oxygen free radicals, cause lysis of erythrocytes by a mechanism associated with the oxidation of oxyhemoglobin to methemoglobin, a process that is inhibited by superoxide dismutase (SOD) and catalase [Weiss S. J.: J. Biol. Chem. 225 (1980) 9912-7]. The chronically inflamed colon is a particularly well endowed with precursors of the superoxide-driven Fenton reaction—including activated leukocytes that make superoxide, heme iron from microscopic hemorrhage with extravasation and lysis of erythrocytes within the subepithelial space, non-heme iron in fecal contents, and low-molecular-weight chelators of iron that can promote Fenton chemistry—all of which may combine to subject the colonic mucosa to oxidative stress. Indeed, the inflamed colonic mucosa, in which the greatest known source of superoxide in the body comes into contact with the greatest concentrations of low-molecular-weight chelate iron normally present within the body, is an ideal reactor for Fenton chemistry.

In addition to classical Fenton chemistry, other processes may be relevant to the pathophysiology of IBD, including lipid oxidation, chloramines generation, and free-radical-mediated carcinogen activation. It has long been reported that hydrogen peroxide combines with hemoglobin to produce a heme-protein-associated radical that peroxidizes polyunsaturated fatty acids and membrane lipids. Monochloramines produced from non-enzymatic reaction of hypochlorous acid (produced by activated neutrophils) and ammonia (produced by gut flora) are also well known for their toxic effects to colonic mucosa. Chloramines are known to exert cytotoxicity through sulfhydryl oxidation, cytochrome inactivation, chlorination of purine bases on DNA, and degradation of proteins and amino acids [Grishman M. B.: Digest Dis. Sciences 33 (1988) 6S-15S]. In particular, chloramines can directly evoke electrolyte and water loss into the lumen of the intestine, an important mechanism for the excessive diarrhea of patients with ulcerative colitis. It has long been proposed by Babbs [Ibid] that free radical mechanisms may be responsible in some individuals for the initiation or promotion of colon carcinoma, a disease that occurs with a high incidence in the patients with ulcerative colitis.

Hence, oxidative stress and free radicals play an important role in the pathogenesis of IBD. The high production of superoxide, and other reactive oxygen species (ROS) such as peroxynitrite, in the gastrointestinal tract increases still more during flare-ups of inflammation, among the causative factors being, e.g., catalase-negative bacteria in colonic flora. It is therefore another object of this invention to provide a new medication for alleviating and treating IBD comprising an antioxidant, preferably a ROS-scavenger.

Animal studies including acetic acid-induced colitis in rodents showed that the treatment with antioxidants like catalase, allopurinol, SOD, and sulfhydryl-protective agents [Keshavarzian A. et al.: Gut 31 (1990) 786-90; Keshavarzian A. et al.: Digest Dis. Sci. 37 (1992) 1866-73], or SOD [Fretland D. J. Gastroenterology 100 (1991) A581] decreased the severity of the inflammation. Colitis induced with trinitrobenzenesulfonic acid in rabbits was also attenuated by SOD [Burakoff R.: Gastroenterology 100 (1991) A565]. Catalase was shown to reduce severity of colitis induced with acetic acid or mitomycin-C in rats [Haydek J.: Gastroenterology 100 (1991) A585]. A phase II uncontrolled human trial using CuZnSOD seemed to provide encouraging results in the treatment of Crohn's disease [Emerit J. et al.: Free Radic. Res. Commun. 12-13 (1991) 563-70].

However, none of the above antioxidant, anti-inflammatory, or immunomodulatory agents offer a universal solution of the serious problems related to IBD, nor do these agents provide a leading compound for possible development of a novel universal medication. PCT/IL03/00878 of the same inventor teaches the preparation and use of a multifunctional steroid compound comprising SOD mimic component. It is therefore still another object of this invention to provide a novel medication affecting IBD through simultaneous multifold mechanisms, comprising both antioxidant effects and anti-inflammatory or immunomodulatory effects.

It is a further object of this invention to provide a method for ameliorating and treating the conditions associated with ulcerative colitis and Crohn's disease.

It is a still further object of this invention to provide a method for inducing remission of an inflammatory bowel disease, and for preventing relapse of an inflammatory bowel disease.

### SUMMARY OF THE INVENTION

The invention relates to the use of a multifunctional compound that comprises in one molecule i) an anti-inflammatory or immunomodulatory component, and ii) at least one SOD mimic component, in the preparation of a medicament for treating inflammatory bowel disease (IBD), comprising ulcerative colitis and Crohn's disease. In a preferred embodiment of the invention, said anti-inflammatory component comprises a derivative of salicylic acid or of cyclopenta[a]phenantrene. Said SOD mimic component should comprise a reactive oxygen species (ROS) scavenging group capable of reacting with superoxide or other reactive oxygen species such as peroxynitrite. ROS group in the use according to the invention may comprise alkenyl; aryl optionally substituted with —OH, —NH₂, or —NHCHO; sulfhydryl or dithiol in oxidized or reduced form; or nitroxide free radical. In a preferred embodiment of the invention, said SOD mimic component comprises a substituted N-oxide free radical or dithiolane-alkanoic structure, preferably of the following formula: 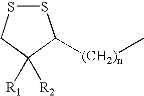
wherein n is an integer from 0 to 6, and R₁ and R₂ are independently selected from C₁-C₄ alkyl. In another preferred embodiment of the invention, said SOD mimic component comprises a substituted N-oxide free radical wherein the N-atom of said N-oxide is a member of 5 to 7 member heterocyclic ring or of 8 to 11 heterobicyclic system. In one preferred embodiment, said anti-inflammatory component preferably comprises a derivative of 4-aminosalicylic acid (ASA) or 5-aminosalicylic acid; in another preferred embodiment said anti-inflammatory or immunomodulatory component comprises a steroid. Said steroid component is selected from corticosteroids, estrogens, progesterones, androgens, analogs thereof, and derivatives thereof. Said steroid component preferably is derived from prednisolone, prednisone, or budesonide.

In a preferred embodiment of the invention, the bi-functional compound for treating IBD has the following structure: 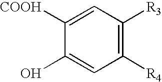
or is an optical isomer, ester, or amide thereof, or a pharmaceutically acceptable salt thereof;
wherein R₃ and R₄ are independently selected from —NHR₅ and —N═R₅, wherein R₅ is a ROS scavenging group, or R₃ and R₄ form together a 5 to 7-membered heterocycle comprising 1 to 2 nitrogen atoms and being substituted with a ROS scavenging group.

In another preferred embodiment of the invention, the bi-functional compound for treating IBD has the following structure: 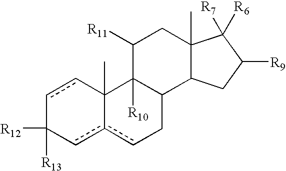
or is an optical isomer, ester, or amide thereof, or a pharmaceutically acceptable salt thereof;
wherein [symbol] is a single or double bond, with the proviso that two double bonds are not adjacent; R⁶ is —H, —OC(O)R⁸ wherein R⁸ is C₁-C₅ alkyl or 5- or 6-membered heteroaryl, or R⁶ and R⁷ together form a substituted N-oxide free radical; R⁷ is —H, or a substituted N-oxide free radical, when possible; R⁹ is —H, —OH, or —CH₃, or R⁶ and R⁹ together form a heterocyclic ring when possible; R¹⁰ is —H or halogen; R¹¹ is —H, ═O, or a substituted N-oxide free radical; R¹² is —H, or ═O, and R¹³, if present, is —H, or R¹² and R¹³ together form a substituted N-oxide free radical; wherein the nitrogen of the N-oxide group in said substituted N-oxide free radical is within a 5- or 6-member ring, which ring may further contain one O or S atom and is substituted with 2 to 4 alkyl groups.

Among preferred bi-functional compounds of the invention are Compounds 1 to 8, having the following structures:

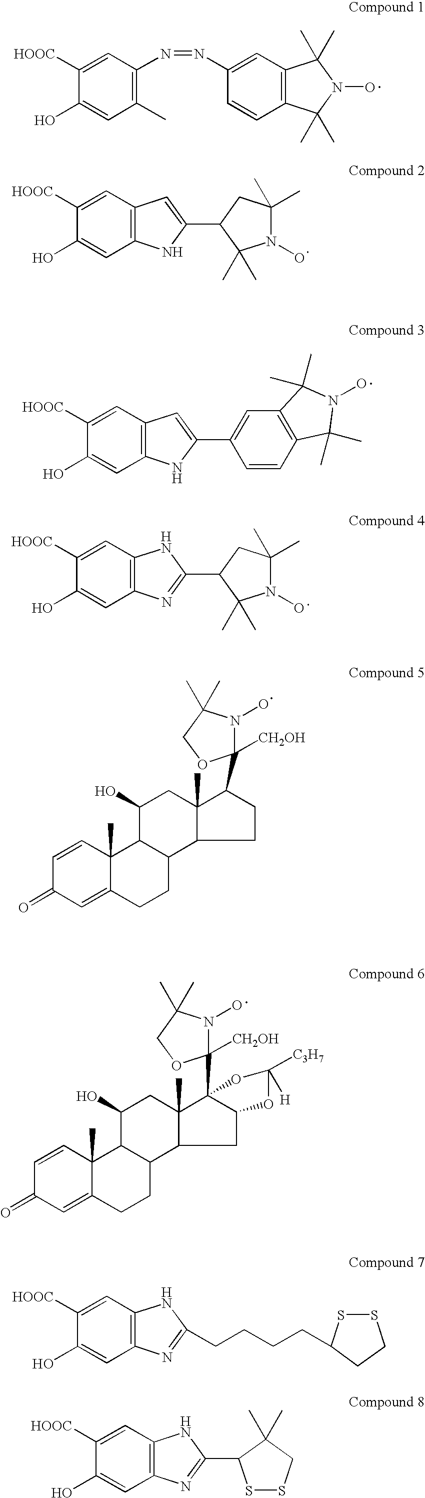

The invention provides a method of treating inflammatory bowel disease in a mammal in need thereof comprising administering to said mammal an effective amount of a bi-functional compound comprising an anti-inflammatory or immunomodulatory component and at least one SOD mimic component. Said method comprises ameliorating the conditions associated with ulcerative colitis or Crohn's disease, inducing remission of inflammatory bowel disease, and preventing relapse of inflammatory bowel disease. Said administration or treatment is selected from oral and parenteral, it may be selected from the group consisting of suppository, by way of injection, and by way of infusion. Said mammal is preferably human.

The invention is also directed to a bi-functional compound comprising an anti-inflammatory component and at least one SOD mimic component of the following structure: 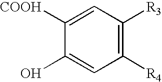
or to an optical isomer, ester, or amide thereof, or to a pharmaceutically acceptable salt thereof; wherein R₃ and R₄ are independently selected from —NHR₅ and —N═R₅, wherein R₅ is a ROS scavenging group, or R₃ and R₄ form together a 5 to 7-membered heterocycle comprising 1 to 2 nitrogen atoms and being substituted with a ROS scavenging group. Said ROS scavenging group in a preferred embodiment of the invention is selected from alkenyl; aryl optionally substituted with —OH, —NH₂, or —NHCHO; sulfhydryl or dithiol in oxidized or reduced form; and nitroxide free radical. In a preferred embodiment of the compound of the invention, said ROS scavenging group comprises the following structure: 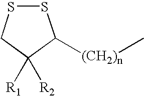
wherein n is an integer from 0 to 6, and R₁ and R₂ are independently selected from C₁-C₄ alkyl. In other preferred embodiment, said ROS scavenging group comprises the a substituted N-oxide free radical wherein the N-atom of said N-oxide is a member of 5 to 7-membered heterocyclic ring or of 8 to 11 heterobicyclic system, wherein said ring or bicyclic system may contain one S or O atom, and may be substituted with 1 to 4 C₁-C₄ alkyl groups.

The invention also provides a pharmaceutical composition comprising a bi-functional compound that comprises an anti-inflammatory or immunomodulatory component and at least one SOD mimic component, and it may further comprise a component selected from carrier, binding agent, stabilizer, adjuvant, diluent, excipient, surfactant, odorant, and a second pharmaceutically active agent selected from antiviral, antifungal, antibacterial, antiseptic, anti-inflammatory or immunomodulatory, antineoplastic, and analgesic. The invention further relates to the use of said compositions as a medicament in treating and preventing symptoms associated with a condition selected from Crohn's disease, ulcerative colitis, enteritis, and inflammatory bowel disease.

### BRIEF DESCRIPTION OF THE DRAWINGS

The above and other characteristics and advantages of the invention will be more readily apparent through the following examples, and with reference to the appended drawings, wherein:

FIG. 1. shows an MS spectrum of Compound 1; and

FIG. 2. shows an NMR spectrum of Compound 7.

### DETAILED DESCRIPTION OF THE INVENTION

Using the acetic acid-induced rat colitis, it has now been found by the inventors that a compound combining in its molecule lipoate-like structure and aminosalicylate-like structure exhibits an activity that is several-fold higher than activities of either 5-aminosalicylic acid (5-ASA) or lipoic acid alone. Other compounds that are anti-inflammatory analogs and comprise in their molecule an antioxidant structure have been synthesized by the inventors, showing remarkable activity in reducing oxidative stress markers in colonic tissues.

A compound according to this invention for treating inflammatory bowel diseases comprises in its molecule both an anti-inflammatory or immunomodulatory component and a ROS-scavenging component. Said anti-inflammatory component preferably comprises a derivative of salicylic acid or of a steroid, and said ROS-scavenging component preferably comprises an N-oxide free radical or a dithiolane-alkanoic structure, such as lipoic acid analog.

As used herein, the term “reactive oxygen species (ROS) scavenger component” refers to a moiety capable of acting as a scavenger of, or reacting with, superoxide (O2—) or other reactive oxygen species (ROS) including hydroxyl radicals, peroxynitrite, hypochlorous acid and hydrogen peroxide. An antioxidant that scavenges, or reacts with, superoxide is termed a “superoxide dismutase mimic” (“SOD-mimic”), superoxide scavenger, or “superoxide dismutase mimetic” (“SOD-mimetic).

A compound according to this invention is a multifunctional anti-inflammatory or immunomodulatory antioxidant. The antioxidant component, such as an ROS scavenger component, may be for example an alkenyl group; aryl group; substituted aryl group, where the aryl group is substituted with, for example, —OH, —NH2, or —NHCHO; sulfhydryl (in a protected form) or dithiol in oxidized or reduced form; or a group that is, or is capable of being converted in vivo into, a sulfhydryl in its oxidized or reduced form. A preferred compound according to this invention is a bi-functional anti-inflammatory SOD-mimetic. The SOD-mimetic component is preferably a nitroxide free radical (NO●) group, or a dithiol structure in its oxidized form, such as lipoic acid analog. In certain embodiments the compounds as described herein may comprise more than one ROS scavenger component.

SOD-mimic-analogs of anti-inflammatory compounds were synthesized by synthetic procedures known in the art. Derivatives of salicylic acid, effective in treating IBD, include sulfasalazine, 5-ASA, 4-ASA, and 4,5-diaminosalicylic acid. In fact, sulfasalazine is a pro-drug with the active ingredient being 5-ASA. Here SOD-mimic analogs of sulfasalazine and ASA were synthesized by standard synthetic procedures, employing diazotization or condensation of the commercially available nitroxides with the appropriate ASA analog. By this way, for example, Compounds 1-4 were obtained. SOD-mimic steroid analogs, comprising prednisolone and budenoside structures, were synthesized by reacting the corresponding steroid with 2-amino-2-methyl propanol under acid catalysis, and converting the resulting ketone-protected steroid derivative to the free radical form (e.g., Compounds 5 and 6). The analogs of ASA comprising a disulfidic anti-ROS group were synthesized by the condensation of lipoic acid and other commercially available (or easily made) dithiols with the corresponding ASA analog (4,5-diaminoacetylsalicylic acid), thus obtaining, e.g., Compounds 7 and 8.

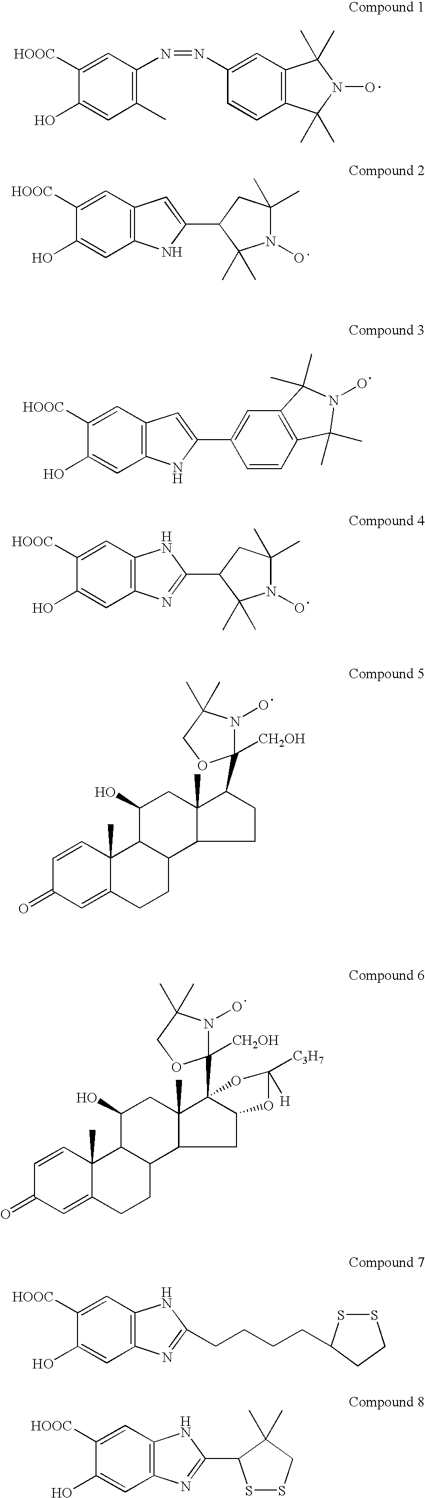

The biological potential of the multifunctional compounds was characterized on animal model, using acetic acid-induced colitis in rats, which is the best animal model for ulcerative colitis. This model comprises all features of ulcerative colitis including histology, eicosanoid production [Sharon P. et al.: Gastroenterology 88 (1985) 55-63], as well as the response to 5-ASA and sulfasalazine [Simmonds N. J. et al.: Gut 33 (suppl) (1992) S7]. Acetic acid-induced colitis is also known to produce excess reactive oxygen species (ROS). At postmortem laparotomy, the macroscopic appearance of the colonic mucosa was checked. Markers of oxidative stress and of inflammation were measured using standard procedures, including measuring tissue protein carbonyl content and thiobarbituric acid reactive substrates. The results clearly indicate that the compounds of the invention possess powerful in vivo activity both when administered prior to colitis induction (to prevent the most severe symptoms or to ameliorate them), and also when administered after the induction in the acetic acid rat model of ulcerative colitis (to treat and alleviate the symptoms).

The multi-mechanism action of the compounds of the invention substantially increases efficacy of the treatment, and moreover, it overcomes the problems of non-responding in about 30-50% of patients to classical therapies. Recent evidence indicates that high therapy failure of the existing drugs for IBD is a matter of metabolic degradation, and possibly of resistance to a specific drug. Ensuring that both components (antioxidant and anti-inflammatory or immunomodulatory) be simultaneously at the site of action by including them both in the same molecule, reduces the possibility of their separate metabolic inactivation (i.e., breakdown before reaching the target site of action). A multifunctional, or bi-functional activity decreases probability of such problems; acting through more than one, or even more than two, mechanisms will help to overcome the resistance. Whenever the term “bi-functional” is used herein for the compounds of the invention, what is intended is “having more than one function”, i.e. multifunctional.

The issue of synergism is highly relevant in the case of compounds of the invention; it was found by the inventors, e.g., that an ASA-like structure comprising a ROS group has an activity that is several-fold higher than either 5-ASA or lipoic acid alone (which was as effective as 5-ASA). If both activities have to be adequately available at the same time and at the same site of action, the best (and maybe the only way) is to have both activities in the same molecule.

As for ROS groups for compounds of the invention, among the preferred configurations are nitroxides. They usually have good pharmacokinetic parameters (absorbability, cell permeability, low toxicity and immunogenicity), they have been shown to be superior to existing native or modified SOD preparations (Miura. Y. et al.: Free Rad. Res. 22 (1995) 209-14; James B. et al.: in Reactive Oxygen Species in Biological Systems: An interdisciplinary approach. Plenum Press, New York (1998) pp 293-313]. Other preferred ROS groups include disulfide structures, such as lipoic acid. Lipoic acid, comprising dithiolane-alkanoic structure, is a naturally (endogenous in humans) occurring antioxidant, being an important co-factor in the multienzyme complexes of various dehydrogenase systems, and combining potent activity against most reactive oxygen species with ideal pharmaceutical characteristics.

As for the anti-inflammatory component in the compounds of the invention, preferred are salicylic acid derivatives, and steroid derivatives. In IBD, as in many other disorders, steroids are “gold standard” for inducing remission, but high and increasing doses are usually required, and even those often fail, leaving surgery as the only choice to manage the disease. The mentioned synergism enables using the modified, multifunctional steroids in lower doses, without developing resistance.

The invention will be further described and illustrated by the following examples.

### EXAMPLES

### Synthesis

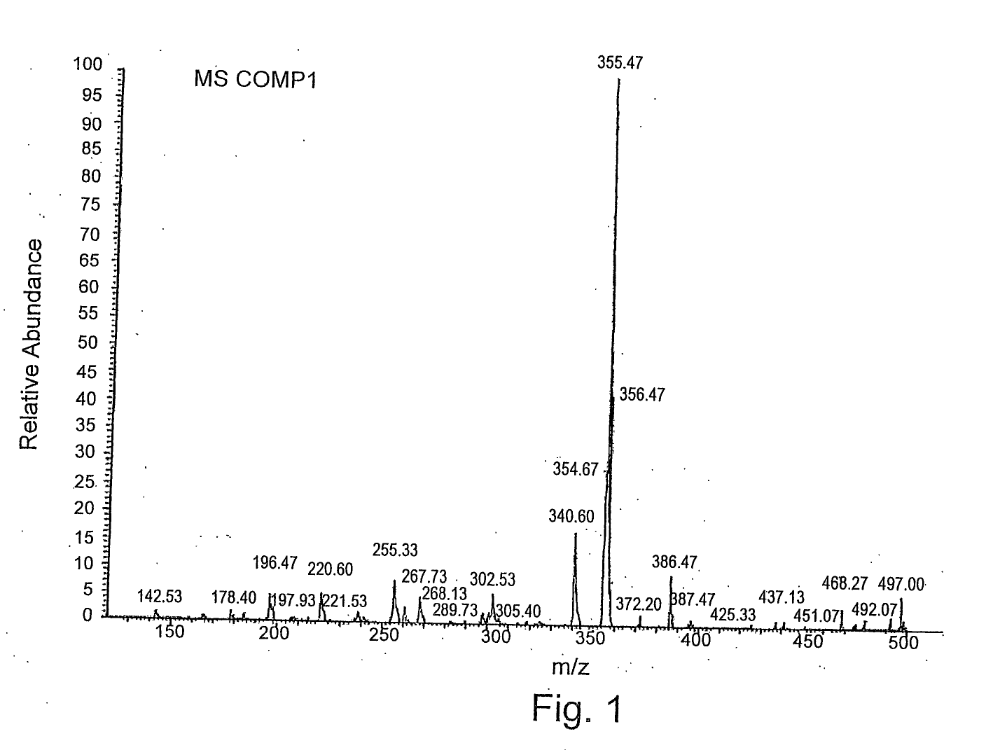

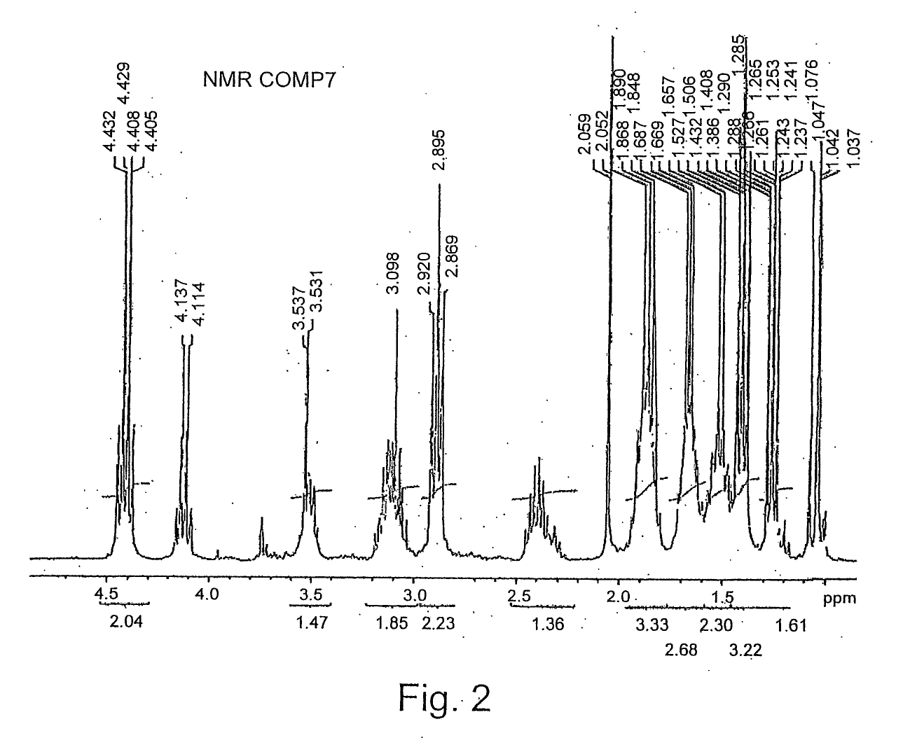

The materials were obtained from Sigma-Aldrich. Compounds 1 to 4 (structures shown above) were synthesized by diazotization or condensation of the commercially available nitroxides with the appropriate ASA analog. Compounds 5 and 6 were synthesized by reacting the steroid with 2-amino-2-methyl propanol under acid catalysis (p-toluenesulfonic acid) using a Dien-Stark apparatus for continuous removal of water. The resulting ketone-protected steroid compound was then converted to the free radical form using tungstate and EDTA-catalyzed oxidation by m-chloroperbenzoic acid oxidation. Compounds 7 and 8 were synthesized by the condensation with disulfidic derivatives. All the compounds were purified and characterized by standard chromatographic, and analytical procedures. In all cases, the synthesized compounds were analyzed also by NMR and MS. FIG. 1, e.g., shows an MS spectrum of Compound 1, and FIG. 2 shows an NMR spectrum of Compound 7.

### Inducing Experimental Colitis in Rats

Although other animal models may be employed if necessary (i.e., deoxycholic acid and trinitrobenzenesulfonic acid-induced colitis), the acetic acid-induced colitis in rats constitutes the best animal model for ulcerative colitis. This model resembles ulcerative colitis in all important features, including histology, eicosanoid production, the response to 5-ASA or sulfasalazine, etc. Acetic acid-induced colitis is also known to produce excess reactive oxygen species (ROS).

Colitis was induced in Sprague-Dawely rats (250-300 g, Hebrew University) using a modification of the acetic acid method [MacPherson B. R. et al.: Digestion 17 (1978) 135-50]. The animals were fasted for 6 hr with access to water, sedated (xylazine), followed by anesthesia with 35 mg/kg intraperitoneal pentobarbital. An infant feeding tube (Pennine Healthcare FT-1608/40) with outside diameter of 2 mm was inserted into the colon to 8 cm, and 2 ml of acetic acid (3% v/v in 0.9% saline) or saline alone (control animals) infused into the lumen. The acetic acid/saline was retained in the colon for 45 seconds, after which fluid was withdrawn. The rats were sacrificed by CO₂ asphyxiation at 24 hr post colitis induction or control treatment. The checked compounds in solutions were adjusted to pH 7.4, and administered either 10 min before or 30 min after the induction of colitis.

### Assessing Colitis

At postmortem laparotomy, 6 cm of colon extending proximally from 2 cm above the anal margin was removed, split longitudinally, pinned out on card, and the macroscopic appearance of the colonic mucosa was scored on a scale ranging from 0-4 as described [Morris G. P. et al.: Gastroenterology 96 (1989) 795-803], wherein (0) is given to no macroscopic change, (1) is scored for mucosal erythema alone, (2) for mild mucosal edema, slight bleeding or small erosions, (3) for moderate edema, bleeding ulcers or erosions, and (4) is scored for severe ulceration/erosions, edema, and tissue necrosis.

Microscopic scoring: Randomly distributed full thickness biopsy specimens were fixed in 10% formyl saline prior to wax embedding, sectioning, and staining with haematoxylin and eosin. Biopsy specimens from all animals were scored according the standard scoring systems [Milar A. D.: Gut 39 (1996) 407-15].

Markers of oxidative stress and inflammation were measured by described procedures, and included the tissue protein carbonyl content (PCC) and the thiobarbituric acid reactive substrates (TBARS) [Nassar T. et al.: Eur. J. Pharmacol. 436 (2002) 111-18].

### Examples 1 to 6

Ulcerative colitis was induced by acetic acid in Sprague-Dawely rats. Vehicle (control healthy rats), acetic acid (AA) alone (ulcerative colitis rats), and acetic acid with either 5-ASA (100 mg/kg) or lipoic acid (LA, 50 mg/kg), or both (AA+5-ASA+LA), as well as AA with compounds 1 and 7 (structures above), were administered in 50 mg/kg, 10 minutes before (pre-treatment) or 30 minutes after (post-treatment) induction of colitis. For each of the seven treatments (six+one control), 6 to 10 animals were used.

These results show that the compounds of the invention possess powerful in vivo activity both when administered prior to colitis induction (prevention model) and (even more promising) when administered post induction in the acetic acid rat model of ulcerative colitis (at least as clearly evident from PCC and TBARS).

| Ex. | Method of | Pre-treatment | | Post-treatment | |
|---|---|---|---|---|---|
| No. | Treatment | PCC | TBARS | PCC | TBARS |
| | Control | 261 ± 27 | 18 ± 4 | 273 ± 21 | 15 ± 3 |
| 1 | AA alone, | 1687 ± 98 | 187 ± 14 | 1587 ± 95 | 163 ± 14 |
| | colitis rats | | | | |
| 2 | AA + | 959 ± 48 | 89 ± 9 | 873 ± 79 | 74 ± 9 |
| | 5-ASA | | | | |
| 3 | AA + | 867 ± 73 | 73 ± 8 | 713 ± 31 | 64 ± 8 |
| | lipoic acid | | | | |
| | (LA) | | | | |
| 4 | AA + | 579 ± 51 | 41 ± 5 | 587 ± 54 | 37 ± 5 |
| | 5-ASA + LA | | | | |
| 5 | AA + | 293 ± 33 | 27 ± 4 | 278 ± 32 | 18 ± 3 |
| | Compound 1 | | | | |
| 6 | AA + | 284 ± 26 | 21 ± 3 | 269 ± 28 | 19 ± 2 |
| | Compound 7 | | | | |

The results in the table show that the rats treated with Compounds 1 or 7 of this invention are statistically indifferent from control healthy rats (P<0.05).

The results show that both Compound 1 and Compound 7 are more active than either of LA or ASA used separately or even simultaneously.

While the invention has been described using some specific examples, many modifications and variations are possible. It is therefore understood that the invention is not intended to be limited in any way other than by the scope of the appended claims.

1. Use of a bi-functional compound comprising
i) an anti-inflammatory or immunomodulatory component, and
ii) at least one SOD mimic component, in the preparation of a medicament for treating inflammatory bowel disease (IBD).

2. Use according to claim 1, wherein said anti-inflammatory or immunomodulatory component comprises a derivative of salicylic acid or of cyclopenta[a]phenantrene.

3. Use according to claim 1, wherein said SOD mimic component comprises a reactive oxygen species (ROS) scavenging group selected from alkenyl; aryl optionally substituted with —OH, —NH₂, or —NHCHO; sulfhydryl or dithiol in oxidized or reduced form; and nitroxide free radical.

4. Use according to claim 1, wherein said SOD mimic component comprises a substituted N-oxide free radical or dithiolane-alkanoic structure.

5. Use according to claim 1, wherein said SOD mimic component comprises the following structure: 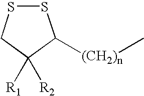
wherein n is an integer from 0 to 6, and R₁ and R₂ are independently selected from C₁-C₄ alkyl.

6. Use according to claim 1, wherein said SOD mimic component comprises a substituted N-oxide free radical wherein the N-atom of said N-oxide is a member of 5 to 7-membered heterocyclic ring or of 8 to 11 heterobicyclic system.

7. Use according to claim 1, wherein said anti-inflammatory component comprises a derivative of 4-aminosalicylic acid (ASA) or 5-aminosalicylic acid.

8. Use according to claim 1, wherein said anti-inflammatory or immunomodulatory component comprises a steroid component.

9. Use according to claim 1, wherein said anti-inflammatory or immunomodulatory component is selected from corticosteroids, estrogens, progesterones, androgens, analogs thereof, and derivatives thereof.

10. Use according to claim 1, wherein said anti-inflammatory or immunomodulatory component is derived from a steroid selected from prednisolone, prednisone, and budesonide.

11. Use according to claim 1, wherein said IBD is ulcerative colitis or Crohn's disease.

12. Use according to claim 1, wherein said bi-functional compound has the following structure: 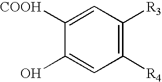
or is an optical isomer, ester, or amide thereof, or a pharmaceutically acceptable salt thereof;
wherein R₃ and R₄ are independently selected from —NHR₅ and —N═R₅, wherein R₅ is a ROS scavenging group, or R₃ and R₄ form together a 5 to 7-membered heterocycle comprising 1 to 2 nitrogen atoms and being substituted with a ROS scavenging group.

13. Use according to claim 1, wherein said bi-functional compound has the following structure: 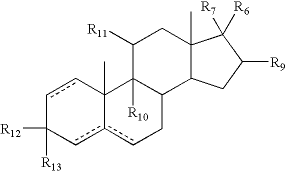
or is an optical isomer, ester, or amide thereof, or a pharmaceutically acceptable salt thereof;
wherein [symbol] is a single or double bond, with the proviso that two double bonds are not adjacent;
R⁶ is —H, —OC(O)R⁸ wherein R⁸ is C₁-C₅ alkyl or 5- or 6-member heteroaryl, or R⁶ and R⁷ together form a substituted N-oxide free radical;
R⁷ is —H, or a substituted N-oxide free radical, when possible;
R⁹ is —H, —OH, or —CH₃, or R⁶ and R⁹ together form a heterocyclic ring, when possible;
R¹⁰ is —H or halogen;
R¹¹ is —H, ═O, or a substituted N-oxide free radical;
R¹² is —H, or ═O; and
R¹³, if present, is —H, or R¹² and R¹³ together form a substituted N-oxide free radical;
wherein the nitrogen of the N-oxide group in said substituted N-oxide free radical is within a 5- or 6-membered ring, which ring may further contain one O or S atom and is substituted with 2 to 4 alkyl groups.

14. Use according to claim 1, wherein said bi-functional compound has one of the following structures: 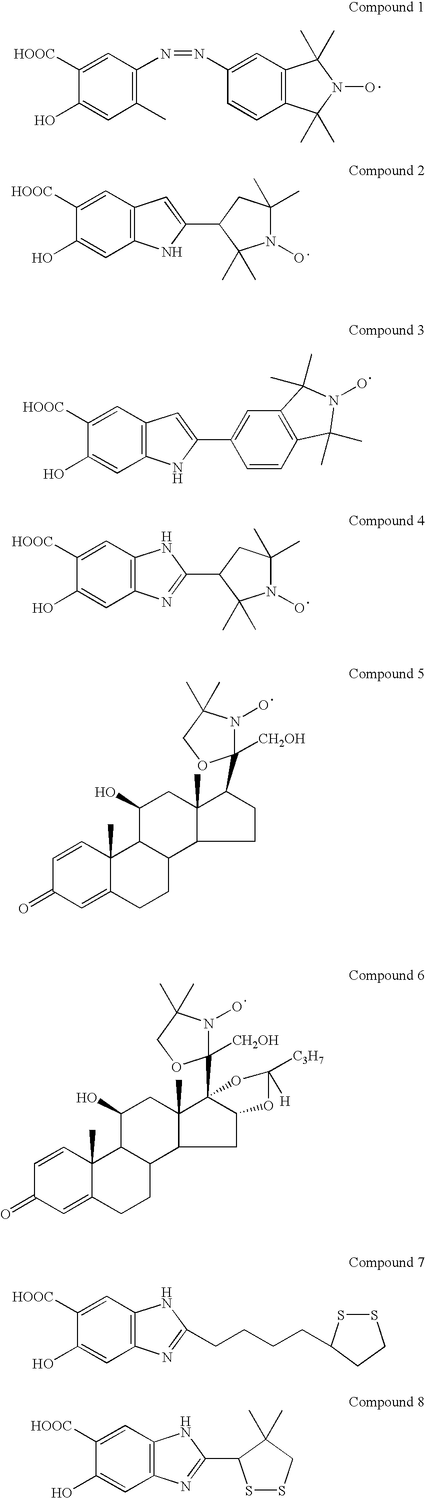
or is an optical isomer, ester, or amide thereof, or a pharmaceutically acceptable salt thereof.

15. A method of treating inflammatory bowel disease in a mammal in need thereof comprising administering to said mammal an effective amount of a bi-functional compound, comprising an anti-inflammatory or immunomodulatory component and at least one SOD mimic component.

16. A method according to claim 15, wherein said bi-functional compound is selected from the group consisting of:
A. said anti-inflammatory or immunomodulatory component is selected from the group consisting of
i. a derivative of salicylic acid or of cyclopenta[a]phenantrene;
ii. a steroid component;
iii. corticosteroids, estrogens, progesterones, androgens, analogs thereof, and derivatives thereof; and
iv. a steroid selected from the group cosnsiting of prednisolone, prednisone, and budesonide
B. said anti-inflammatory component comprises a derivative of 4-aminosalicylic acid (ASA) or 5-aminosalicylic acid
C. said SOD mimic component selected from the group consisting of:
i. a reactive oxygen species (ROS) scavenging group selected from alkenyl; aryl optionally substituted with —OH, —NH₂, or —NHCHO; sulfhydryl or dithiol in oxidized or reduced form; and nitroxide free radical;
ii. a substituted N-oxide free radical or dithiolane-alkanoic structure;
iii. the following structure: 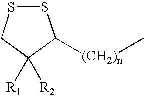
wherein n is an integer from 0 to 6, and R₁ and R₂ are independently selected from C₁-C₄ alkyl; and
iv. a substituted N-oxide free radical wherein the N-atom of said N-oxide is a member of 5 to 7-membered heterocyclic ring or of 8 to 11 heterobicyclic system;
D. bi-functional compound has one of the following structures:
i. 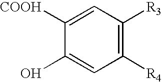
or is an optical isomer, ester, or amide thereof, or a pharmaceutically acceptable salt thereof;
wherein R₃ and R₄ are independently selected from —NHR₅ and —N═R₅, wherein R₅ is a ROS scavenging group, or R₃ and R₄ form together a 5 to 7-membered heterocycle comprising 1 to 2 nitrogen atoms and being substituted with a ROS scavenging group;
ii. 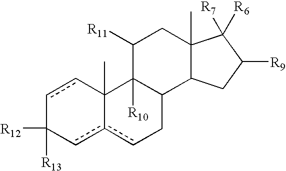
or is an optical isomer, ester, or amide thereof, or a pharmaceutically acceptable salt thereof;
wherein [symbol] is a single or double bond, with the proviso that two double bonds are not adjacent;
R⁶ is —H, —OC(O)R⁸ wherein R⁸ is C₁-C₅ alkyl or 5- or 6-member heteroaryl, or R⁶ and R⁷ together form a substituted N-oxide free radical;
R⁷ is —H, or a substituted N-oxide free radical, when possible;
R⁹ is —H, —OH, or —CH₃, or R⁶ and R⁹ together form a heterocyclic ring, when possible;
R¹⁰ is —H or halogen;
R¹¹ is —H, ═O, or a substituted N-oxide free radical;
R¹² is —H, or ═O; and
R¹³, if present, is —H, or R¹² and R¹³ together form a substituted N-oxide free radical;
wherein the nitrogen of the N-oxide group in said substituted N-oxide free radical is within a 5- or 6-membered ring, which ring may further contain one O or S atom and is substituted with 2 to 4 alkyl groups;
iii. 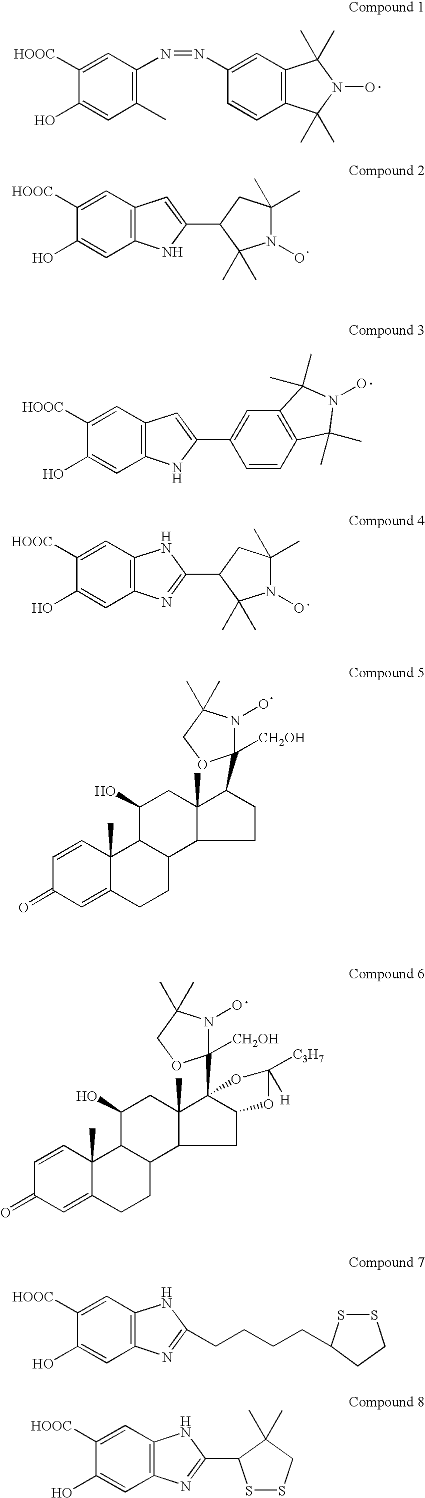
or is an optical isomer, ester, or amide thereof, or a pharmaceutically acceptable salt thereof; and
E. mixtures thereof.

17. A method according to claim 15, comprising ameliorating the conditions associated with ulcerative colitis or Crohn's disease.

18. A method according to claim 15, comprising inducing remission of inflammatory bowel disease.

19. A method according to claim 15, comprising preventing relapse of inflammatory bowel disease.

20. A method according to claim 15, wherein said administration or treatment is selected from oral and parenteral.

21. A method according to claim 15, wherein said administration or treatment is selected from the group consisting of suppository, by way of injection, and by way of infusion.

22. A method according to claim 15, wherein said mammal is human.

23. A bi-functional compound comprising an anti-inflammatory component and at least one SOD mimic component of the following structure: 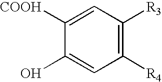
or an optical isomer, ester, or amide thereof, or a pharmaceutically acceptable salt thereof;
wherein R₃ and R₄ are independently selected from —NHR₅ and —N═R₅, wherein R₅ is a ROS scavenging group, or R₃ and R₄ form together a 5 to 7-membered heterocycle comprising 1 to 2 nitrogen atoms and being substituted with a ROS scavenging group.

24. A compound according to claim 23, wherein said ROS scavenging group is selected from alkenyl; aryl optionally substituted with —OH, —NH₂, or —NHCHO; sulfhydryl or dithiol in oxidized or reduced form; and nitroxide free radical.

25. A compound according to claim 23, wherein said ROS scavenging group comprises the following structure: 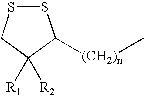
wherein n is an integer from 0 to 6, and R₁ and R₂ are independently selected from C₁-C₄ alkyl.

26. A compound according to claim 23, wherein said ROS scavenging group comprises a substituted N-oxide free radical wherein the N-atom of said N-oxide is a member of 5 to 7-membered heterocyclic ring or of 8 to 11 heterobicyclic system, wherein said ring or bicyclic system may contain one S or O atom, and may be substituted with 1 to 4 C₁-C₄ alkyl groups.

27. A bi-functional compound comprising an anti-inflammatory or immunomodulatory component and at least one SOD mimic component of the following structures: 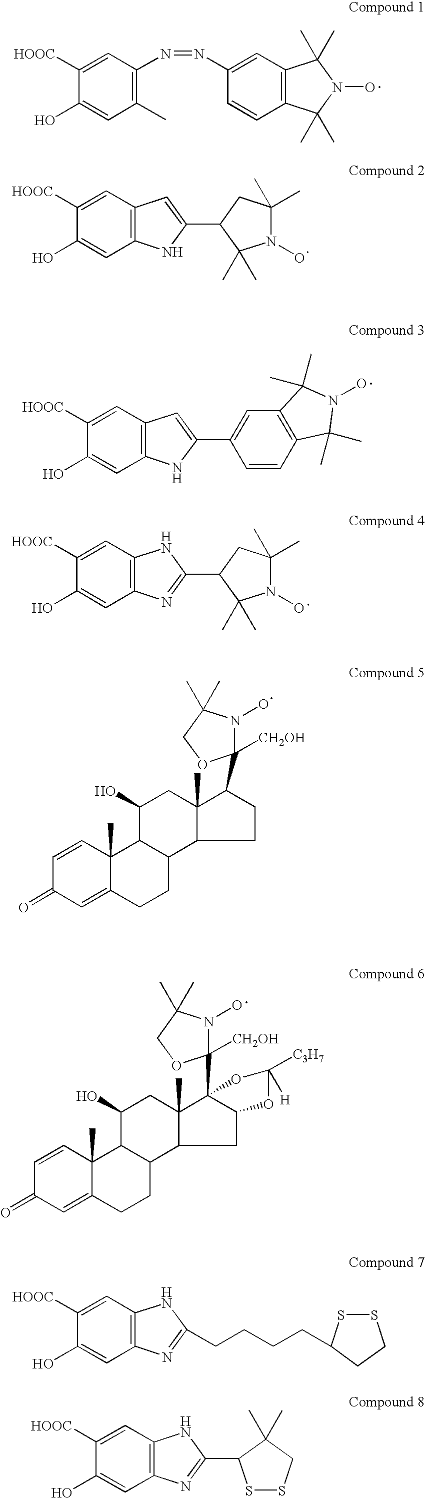
or an optical isomer, ester, or amide thereof, or a pharmaceutically acceptable salt thereof.

28. A pharmaceutical composition comprising a bi-functional compound that comprises an anti-inflammatory or immunomodulatory component and at least one SOD mimic component.

29. A pharmaceutical composition comprising a bi-functional compound selected from the group consisting of an anti-inflammatory component and at least one SOD mimic component of the following structures: 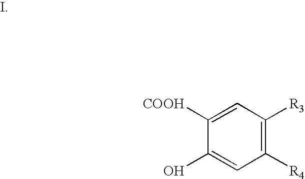
or an optical isomer, ester, or amide thereof, or a pharmaceutically acceptable salt thereof;
wherein R₃ and R₄ are independently selected from —NHR₅ and —N═R₅, wherein R₅ is a ROS scavenging group, or R₃ and R₄ form together a 5 to 7-membered heterocycle comprising 1 to 2 nitrogen atoms and being substituted with a ROS scavenging group;
a. said ROS scavenging group is selected from the group consisting of:
i. alkenyl; aryl optionally substituted with —OH, —NH₂, or —NHCHO; sulfhydryl or dithiol in oxidized or reduced form; and nitroxide free radical;
ii. the following structure: 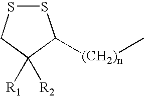
wherein n is an integer from 0 to 6, and R₁ and R₂ are independently selected from C₁-C₄ alkyl; and
iii. a substituted N-oxide free radical wherein the N-atom of said N-oxide is a member of 5 to 7-membered heterocyclic ring or of 8 to 11 heterobicyclic system, wherein said ring or bicyclic system may contain one S or O atom, and may be substituted with 1 to 4 C₁-C₄ alkyl groups; and
II. 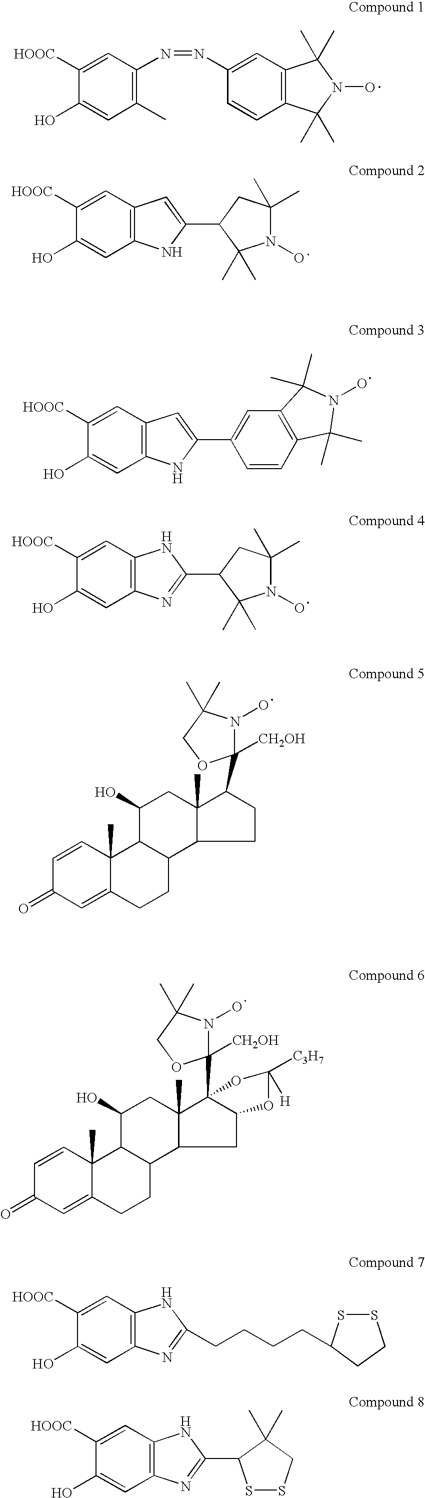
or an optical isomer, ester, or amide thereof, or a pharmaceutically acceptable salt thereof.

30. A pharmaceutical composition according to claim 29, further comprising a component selected from carrier, binding agent, stabilizer, adjuvant, diluent, excipient, surfactant, odorant, and a second pharmaceutically active agent.

31. A pharmaceutical composition according to claim 30, wherein said second active agent is selected from antiviral, antifungal, antibacterial, antiseptic, anti-inflammatory or immunomodulatory, antineoplastic, and analgesic.

32. A pharmaceutical composition according to claim 29 for use as a medicament in treating and preventing symptoms associated with a condition selected from Crohn's disease, ulcerative colitis, enteritis, and inflammatory bowel disease.

33. A pharmaceutical composition according to claim 28, further comprising a component selected from carrier, binding agent, stabilizer, adjuvant, diluent, excipient, surfactant, odorant, and a second pharmaceutically active agent.

34. A pharmaceutical composition according to claim 28 for use as a medicament in treating and preventing symptoms associated with a condition selected from Crohn's disease, ulcerative colitis, enteritis, and inflammatory bowel disease.
